SEQUENCE LISTING (66 sequences)

SEQ ID NO: 1 - DNA, 24 bp, Artificial Sequence
  probe
tgaaagaggaaagcggaatttcca

SEQ ID NO: 2 - DNA, 21 bp, Artificial Sequence
  probe
gtagaggggggtagaattcca

SEQ ID NO: 3 - DNA, 21 bp, Artificial Sequence
  probe
agagggtggtggaatttcctg

SEQ ID NO: 4 - DNA, 25 bp, Artificial Sequence
  probe
gtatgggagaggatggtagaattcc

SEQ ID NO: 5 - DNA, 19 bp, Artificial Sequence
  probe
gcagccccttgggaatgta

SEQ ID NO: 6 - DNA, 23 bp, Artificial Sequence
  probe
ggtctgtcaagtcggatgtgaaa

SEQ ID NO: 7 - DNA, 21 bp, Artificial Sequence
  probe
agggctgcaagctagcgatag

SEQ ID NO: 8 - DNA, 19 bp, Artificial Sequence
  probe
acatgcacaggacgcgtct

SEQ ID NO: 9 - DNA, 19 bp, Artificial Sequence
  probe
aacacaggtgctgcatggc

SEQ ID NO: 10 - DNA, 19 bp, Artificial Sequence
  probe
cttgacatcccgatgcccg

SEQ ID NO: 11 - DNA, 24 bp, Artificial Sequence
  probe
tctgcaactcgactacatgaagct

SEQ ID NO: 12 - DNA, 23 bp, Artificial Sequence
  probe
gtcgcgaggctaagctaatctct

SEQ ID NO: 13 - DNA, 22 bp, Artificial Sequence
  probe
ggtgacggcaagctaatctctt

SEQ ID NO: 14 - DNA, 24 bp, Artificial Sequence
  probe
tcgacacactatcattaacgtgat

SEQ ID NO: 15 - DNA, 19 bp, Artificial Sequence
  probe
gttacagccggcgatgtcc

SEQ ID NO: 16 - DNA, 19 bp, Artificial Sequence
  probe
acccagtgtgwttcgwcac

SEQ ID NO: 17 - DNA, 21 bp, Artificial Sequence
  probe
acttcggtgcatcatcgttag

SEQ ID NO: 18 - DNA, 19 bp, Artificial Sequence
  probe
gcgtgtaggtgtgtgttcc

SEQ ID NO: 19 - DNA, 20 bp, Artificial Sequence
  probe
ccggtttaagcgtgtaggta

SEQ ID NO: 20 - DNA, 22 bp, Artificial Sequence
  probe
cctgaatacatagggtaatgaa

SEQ ID NO: 21 - DNA, 23 bp, Artificial Sequence
  probe
caaacagactttgatccggagat

SEQ ID NO: 22 - DNA, 22 bp, Artificial Sequence
  probe
acccggtatccaatggatacta

SEQ ID NO: 23 - DNA, 23 bp, Artificial Sequence
  probe
catgacgtgaatacatagcgtca

SEQ ID NO: 24 - DNA, 24 bp, Artificial Sequence
  probe
agcagtaaggtgtgatatgagtca

SEQ ID NO: 25 - DNA, 19 bp, Artificial Sequence
  probe
cccagtgtgattcgtcaca

SEQ ID NO: 26 - DNA, 20 bp, Artificial Sequence
  probe
tccggttgcttatcaacgct

SEQ ID NO: 27 - DNA, 19 bp, Artificial Sequence
  probe
cgcacaagcggtggagcat

SEQ ID NO: 28 - DNA, 20 bp, Artificial Sequence
  probe
cgcacaagcgtggagcatgt

SEQ ID NO: 29 - DNA, 18 bp, Artificial Sequence
  probe
gcgaagtggttgatgcca

SEQ ID NO: 30 - DNA, 18 bp, Artificial Sequence
  probe
ggctgataccatgcttcc

SEQ ID NO: 31 - DNA, 23 bp, Artificial Sequence
  probe
agcaagctagtttactagcgaag

SEQ ID NO: 32 - DNA, 25 bp, Artificial Sequence
  probe
cttttaagtgaggtgtgaaatcccc

SEQ ID NO: 33 - DNA, 25 bp, Artificial Sequence
  probe
gactgggtaactagagtactttagg

SEQ ID NO: 34 - DNA, 21 bp, Artificial Sequence
  probe
ggtaccttttaacgctgaggt

SEQ ID NO: 35 - DNA, 19 bp, Artificial Sequence
  probe
tccggaacgttaytaacgc

SEQ ID NO: 36 - DNA, 21 bp, Artificial Sequence
  probe
gagacacaacactgagacacg

SEQ ID NO: 37 - DNA, 25 bp, Artificial Sequence
  probe
aagcgaatccttttaaagccggtct

SEQ ID NO: 38 - DNA, 24 bp, Artificial Sequence
  probe
aagcgaatccttaaaagccggtct

SEQ ID NO: 39 - DNA, 23 bp, Artificial Sequence
  probe
acctggttttgacatgtgcggaa

SEQ ID NO: 40 - DNA, 24 bp, Artificial Sequence
  probe
agaaccttacctggtcttgacatg

SEQ ID NO: 41 - DNA, 24 bp, Artificial Sequence
  probe
agccaatccagaaaccctatctca

SEQ ID NO: 42 - DNA, 25 bp, Artificial Sequence
  probe
ggagcatgtggtttattcgaagcaa

SEQ ID NO: 43 - DNA, 23 bp, Artificial Sequence
  probe
gctgcaactcgctacatgaagtc

SEQ ID NO: 44 - DNA, 23 bp, Artificial Sequence
  probe
gctgcaactcgcctacatgaagt

SEQ ID NO: 45 - DNA, 23 bp, Artificial Sequence
  probe
gcatcaatcactagcggatatga

SEQ ID NO: 46 - DNA, 19 bp, Artificial Sequence
  probe
tttagatagcgtaccttca

SEQ ID NO: 47 - DNA, 21 bp, Artificial Sequence
  probe
tgcagcaattgaaggtactga

SEQ ID NO: 48 - DNA, 19 bp, Artificial Sequence
  probe
gccatctaatattcaaaca

SEQ ID NO: 49 - DNA, 22 bp, Artificial Sequence
  probe
tgcagcagtagaaggaactgat

SEQ ID NO: 50 - DNA, 19 bp, Artificial Sequence
  probe
gagggaacagatcttgaat

SEQ ID NO: 51 - DNA, 19 bp, Artificial Sequence
  probe
ytctgcagttgaaggtact

SEQ ID NO: 52 - DNA, 22 bp, Artificial Sequence
  probe
atctgacatccaaactgcagtc

SEQ ID NO: 53 - DNA, 19 bp, Artificial Sequence
  probe
aagaaataacggtggcgga

SEQ ID NO: 54 - DNA, 22 bp, Artificial Sequence
  probe
gcaaayctagrcagtgttccag

SEQ ID NO: 55 - DNA, 24 bp, Artificial Sequence
  probe
accgtataacaataaaccgcatgg

SEQ ID NO: 56 - DNA, 20 bp, Artificial Sequence
  probe
cgtataacaatcgaaaccgc

SEQ ID NO: 57 - DNA, 19 bp, Artificial Sequence
  probe
gttaagtcccgcaacggcg

SEQ ID NO: 58 - DNA, 25 bp, Artificial Sequence
  probe
agaaaccgcatggtttctatttgaa

SEQ ID NO: 59 - DNA, 22 bp, Artificial Sequence
  probe
aaaaattccagcagacatccgt

SEQ ID NO: 60 - DNA, 24 bp, Artificial Sequence
  probe
cttgcaccatgacaaacaccatca

SEQ ID NO: 61 - DNA, 23 bp, Artificial Sequence
  probe
tgatgtagatctatcccagctga

SEQ ID NO: 62 - DNA, 23 bp, Artificial Sequence
  probe
ggaacaaatcccagctgatattc

SEQ ID NO: 63 - DNA, 24 bp, Artificial Sequence
  probe
catcctgaaattggagaagactta

SEQ ID NO: 64 - DNA, 24 bp, Artificial Sequence
  probe
tggaaaaattattggctgatgtgg

SEQ ID NO: 65 - DNA, 23 bp, Artificial Sequence
  probe
ggaactattggcagatgtcacta

SEQ ID NO: 66 - DNA, 20 bp, Artificial Sequence
  probe
catgacaagcaccatcaarc

(19) United States
(12) Patent Application Publication
(10) Pub. No.: US 2006/0204995 A1
(43) Pub. Date: Sep. 14, 2006

(54) Method of designing probe set, probe set designed by the method, microarray comprising the probe set, computer readable medium recorded thereon program to execute the method, and method of identifying target sequence using the probe set

(76) Inventors: Ji-young Oh, Suwon-si (KR); Kyu-sang Lee, Suwon-si (KR); Tae-joon Kwon, Seoul (KR)

Correspondence Address: CANTOR COLBURN, LLP, 55 GRIFFIN ROAD SOUTH, BLOOMFIELD, CT, 06002, US

(21) Appl. No.: 11/370,433

(22) Filed: Mar. 8, 2006

(30) Foreign Application Priority Data
Mar. 8, 2005 (KR) 10-2005-0019065
Feb. 28, 2006 (KR) 10-2006-0019499

(51) Int. Cl. C12Q 1/68; G06F 19/00; C07H 21/04; C12M 1/34

(52) U.S. Cl. 435/6; 435/287.2; 536/24.3; 702/20

(57) ABSTRACT

A method of designing a probe set for identification of target sequences is provided. Also, a probe set designed by the method, a microarray including the probe set, a computer readable medium recorded thereon a program to execute the method, and a method of identifying target sequences using the probe set are provided. Accordingly, a probe set which can rapidly identify a number of target sequences and accurately identify target sequences even when two or more target sequences coexist in a sample can be readily designed.

### BACKGROUND OF THE INVENTION

This application claims the benefit of Korean Patent Application Nos. 10-2005-0019065 and 10-2006-0019499, filed on Mar. 8, 2005 and Feb. 28, 2006, in the Korean Intellectual Property Office, the disclosure of which is incorporated herein in its entirety by reference.

1. Field of the Invention

The present invention relates to a method of designing a probe set for identification of target sequences, a probe set designed by the method, a microarray comprising the probe set, a computer readable medium recorded thereon a program to execute the method, and a method of identifying a target sequence using the probe set.

2. Description of the Related Art

A microarray is a substrate on which polynucleotides are immobilized at fixed locations. Such a microarray is well known in the art and examples thereof can be found in, for example, U.S. Pat. Nos. 5,445,934 and 5,744,305. Also, it is known that the microarray is generally manufactured using photolithography. When using photolithography, the polynucleotide microarray can be manufactured by repeatedly exposing an energy source to a discrete known region on a substrate, on which a monomer protected by a removable group is coated, to remove the protecting group, and coupling the deprotected monomer with another monomer protected by the removable group. In this case, the polynucleotide immobilized on a microarray is synthesized by extending monomers of the polynucleotide one by one. Alternatively, when using a spotting method, a microarray is formed by immobilizing previously-synthesized polynucleotides at fixed locations. Such methods of manufacturing a microarray are disclosed in, for example, U.S. Pat. Nos. 5,744,305, 5,143,854, and 5,424,186. These documents related to microarrays and methods of manufacturing the same are incorporated herein in their entirety by reference.

A polynucleotide (also called “a probe”, “a probe nucleic acid”, or “a probe polynucleotide”) which is immobilized on the microarray can be specifically hybridized with a target nucleic acid, and thus is used to detect and identify the target sequence. A conventional probe DNA is selected by establishing criterions for selecting a probe DNA for each target sequence and selecting DNA sequences which meet the criterions. The selected DNA sequences are investigated to determine whether they meet the above criterions and other requirements and the most desirable probe sequence is selected. The criterions may include a length of the probe, a Tm (a temperature at which 50% of double-strand DNA molecules are dissociated into two single strand) of the probe, and sequence homology with other DNAs. When candidate probe DNAs which meet the criterions are selected, whether they are unique only to the target sequence and whether they are easily cross-hybridized are investigated through Tm and sequence homology. The most desirable probe DNA among candidate probe DNAs which meet the criterions is selected as a sequence specifically bonding to the target DNA sequence. However, since this method of designing a probe set selects as a probe only a specific sequence which is hybridized with the target sequence but does not cross-hybridize with other sequences, it is difficult to design a specific probe when sequence homology between target sequences is high or the number of target sequences to be identified is large.

For example, to identify species of bacteria in a sample, a consensus sequence of a plurality of bacteria, in particular a 16S rRNA site has been conventionally used. That is, common sequences at the 16S rRNA site of a plurality of bacteria are used as primers and unique sequences of the respective bacteria are used as probes. Such a method can be used to identify several species of bacteria, but is limited in identification of ten or more species of bacteria since the 16S rRNA site is highly conserved. For example, in the case of total 71 species including 37 species of bacteria related to sepsis and 34 species of bacteria related to contamination during culturing blood or bacteremia, the sequence homology of 14 species of bacteria is 100% at 16S rRNA sequence of 1,002 bp. The sequence homology of 97% of 71 species of bacteria is 70% or more and an average sequence homology of 71 species of bacteria is 83%, indicating that 16S rRNA site is highly conserved. Thus, when probes for identification of 71 species of bacteria described above are designed using a conventional method, only 12 species can be identified when designing probes such that the homology between probes is 80% or less.

23S rRNA, which is another gene for identifying species of bacteria, shows somewhat of a difference between species, and thus can identify more species than when using 16S rRNA. However, since 23S rRNA sequences of many species are not known, additional costs to identify the sequences are incurred. For example, at least half of about 2,600 bp of 23S rRNA sequences are known for only 43 species among 71 species of bacteria. It was reported that 30 species of bacteria were identified using 23S rRNA sequence [“Rapid diagnosis of bacteremia by universal amplification of 23S ribosomal DNA followed by hybridization to an oligonucleotide array, JOURNAL OF CLINICAL MICROBIOLOGY, February 2000, pp. 781-788]. However, 23S rRNA sequence is not disclosed in the document and it is still impossible to identify 30 or more species.

In a word, the conventional method of designing probes capable of discriminating a number of species have the following limitations. First, it is difficult to acquire known sequences of the same site in a number of species other than 16S rRNA. Second, since 16S rRNA has a highly conserved sequence, it is difficult to find sequences for discriminating species in the same genus. Third, when species have relatively different sequences at a specific site on gene or genome, a separate experiment should be conducted to obtain sequences of all species, resulting in an increase in costs and delay of development.

The inventors of the present invention found that a probe set capable of identifying 30 or more target sequences by comparing a consensus sequence of target sequences to form groups, each of which consists of target sequences having an identical sequence, selecting a target sequence specific probe when a group consists of one target sequence, selecting a group probe when a group consists of two or more target sequences, and performing the above-described process using another consensus sequence of target sequences of groups consisting of two or more target sequences, and thus completed the present invention.

### SUMMARY OF THE INVENTION

The present invention provides a method of designing a probe set used for identification of a target sequence.

The present invention also provides a probe set designed according to the method.

The present invention also provides a microarray including the probe set.

The present invention also provides a computer readable medium recorded thereon a program to execute the method.

The present invention also provides a method of identifying a target sequence using the probe set.

According to one aspect of the present invention, there is provided a method of designing a probe set for identification of a target sequence, including: (a) comparing a consensus sequence of target sequences to form groups, each of which consists of target sequences which include a polynucleotide contained in the consensus sequence and meeting a predetermined criterion; (b) selecting an oligonucleotide specifically binding to the polynucleotide meeting the predetermined criterion as a target sequence specific probe when one of the groups formed in the operation (a) consists of one target sequence; (c) selecting an oligonucleotide specifically binding to the polynucleotide meeting the predetermined criterion as a group probe when one of the groups formed in the operation (a) consists of two or more target sequences; and (d) performing operations (a) to (c) on the groups formed in the operation (a) consisting of two or more target sequences using a consensus sequence other than the consensus sequence used in the operation (a) until there are no groups consisting of two or more target sequences.

In the method of designing a probe set, the predetermined criterion may be at least one selected from the group consisting of a sequence homology, a base length, a hybridization melting point (Tm), a difference between hybridization melting points (ΔTm), a GC content, self-alignment, a mutation position, a repeating sequence level, and a base composition at the 3′ end.

In the method of designing a probe set, the predetermined criterion may be a homology of 100% for polynucleotides of the same group and 90% or less for polynucleotides of different groups.

In the method of designing a probe set, the consensus sequence may be 16S rRNA, 23S rRNA, sodA, gyrA, groEL, or rpoB.

According to another aspect of the present invention, there is provided a probe set designed using the method.

According to another aspect of the present invention, there is provided a microarray for identification of target sequences, in which the probe set is immobilized on a substrate.

The substrate may be coated with an active group selected from the group consisting of amino-silane, poly-L-lysine, and aldehyde.

The substrate may be a silicon wafer, glass, quartz, metal, or plastic.

According to another aspect of the present invention, there is provided a computer readable medium recorded thereon a program to execute the method.

According to another aspect of the present invention, there is provided a method of identifying target sequences using the probe set.

The method of identifying a target sequence may include: applying a sample including target sequences on the microarray described above; hybridizing the target sequences with the probe set; washing the microarray to remove a non-specific reaction; and detecting a fluorescent signal due to hybrid formation.

### BRIEF DESCRIPTION OF THE DRAWINGS

The above and other features and advantages of the present invention will become more apparent by describing in detail exemplary embodiments thereof with reference to the attached drawings in which:

FIG. 1 is a flow chart of a method of designing a probe set according to an embodiment of the present invention;

FIG. 2 is a schematic diagram of a method of designing a probe set using two consensus sequences according to an embodiment of the present invention;

FIG. 3 is a schematic diagram of a method of designing a probe set using two consensus sequences according to another embodiment of the present invention;

FIG. 4 is a schematic diagram of a method of designing a probe set using three consensus sequences according to an embodiment of the present invention;

FIG. 5 is a schematic diagram of a method of designing a probe set using three consensus sequences according to another embodiment of the present invention;

FIG. 6 is a spotting arrangement of a microarray including the probe set designed by the method illustrated in FIG. 2 and a method of identifying target sequences using the microarray;

FIG. 7 is a spotting arrangement of a microarray including the probe set designed by the method illustrated in FIG. 3 and a method of identifying target sequences using the microarray;

FIG. 8 is a spotting arrangement of a microarray including the probe set designed by the method illustrated in FIG. 4 and a method of identifying a target sequence using the microarray; and

FIG. 9 is a spotting arrangement of a microarray including the probe set designed by the method illustrated in FIG. 5 and a method of identifying a target sequence using the microarray.

### DETAILED DESCRIPTION OF THE INVENTION

The present invention will now be described more fully with reference to the accompanying drawings, in which exemplary embodiments of the invention are shown.

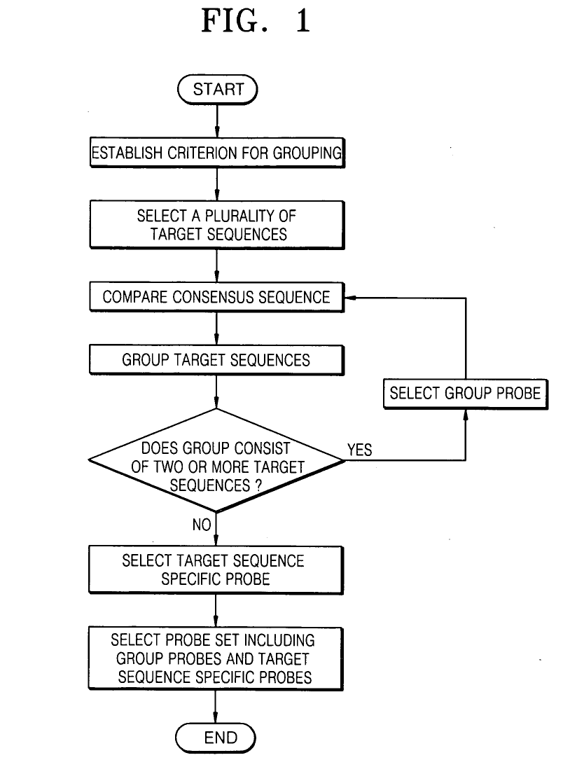

FIG. 1 is a flow chart of a method of designing a probe set according to an embodiment of the present invention.

A method of designing a probe set according to an embodiment of the present invention includes an operation (a) of comparing a consensus sequence of target sequences to form groups, each of which consists of target sequences which include a polynucleotide contained in the consensus sequence and meeting a predetermined criterion.

As used herein, the term “the target sequence” refers to a polynucleotide selected to be identified by binding to a probe. Examples of the target sequence include genome DNA, a DNA fragment cleaved by a restriction enzyme, and a PCR product. A genome DNA fragment obtained by amplifying a specific region of genome DNA through a polymerase chain reaction (PCR) is generally used. The method of the present embodiment is to design a probe set which can be applied to two or more target sequences.

As used herein, the term “the consensus sequence” refers to a polynucleotide which is located at the same site in given target sequences and has an identical or similar base sequence. The consensus sequence may be any gene of given target sequences. For example, the consensus sequence compared in the operation (a) may be 16S rRNA, 23S rRNA, sodA, gyrA, groEL, or rpoB.

In the method of designing a probe set, the predetermined criterion may be a typical criterion for probe selection. That is, the criterion may be at least one selected from the group consisting of a sequence homology, a base length, a hybridization melting point (Tm), a difference between hybridization melting points (ΔTm), a GC content, self-alignment, a mutation position, a repeating sequence level, and a base composition at the 3′ end. The predetermined criterion may be a homology of 100% for polynucleotides of the same group and 90% or less for polynucleotides of different groups, a base length of 18-25 bp, a hybridization temperature of 72-76C, a GC content of 30-70%, and a base composition at the 3′ end of G or C.

The method of designing a probe set also includes the operation (b) of selecting an oligonucleotide specifically binding to the polynucleotide meeting the predetermined criterion as a target sequence specific probe when one of the groups formed in the operation (a) consists of one target sequence.

The probe may be selected according to the criterion described above using conventional methods. That is, the criterion for selecting a probe DNA is established, DNA sequences meeting the criterion are selected, whether the selected DNA sequences meet the criterion and other requirements is investigated, and a most preferable sequence is selected as the probe DNA. Once candidate probe DNAs meeting the criterion are selected, the most preferable DNA among the candidate probe DNAs is selected as a probe DNA specifically binding to a target DNA. Two or more probe DNAs may be selected as long as they can specifically bind to the target DNA.

The method of designing a probe set also includes the operation (c) of selecting an oligonucleotide specifically binding to the polynucleotide meeting the predetermined criterion as a group probe when one of the groups formed in the operation (a) consists of two or more target sequences.

The method of designing a probe set also includes the operation (d) of performing the operations (a) to (c) on target sequences of the groups formed in the operation (a) consisting of two or more target sequences using a consensus sequence other than the consensus sequence used in the operation (a) until there are no groups consisting of two or more target sequences.

The consensus sequence compared in the operation (d) may be any gene of target sequences of groups, each of which consists of two or more target sequences. The consensus sequence compared in the operation (d) may be selected from consensus sequences other than the consensus sequence compared in the operation (a). For example, when 16S rRNA is compared in the operation (a), the consensus sequence of the operation (d) may be selected from 23S rRNA, sodA, gyrA, groEl, and rpoB. Also, it is not necessary that consensus sequences compared in the respective groups are identical. For example, when 16S rRNA is compared in the operation (a), 23S rRNA can be compared in a group and sodA can be compared in another group in the operation (d).

The present operation (d) is performed until there are no groups consisting of two or more target sequences, i.e., all groups consist of one target sequence and the respective target sequence specific probes for the respective target sequences are selected.

The obtained group probes and target sequence specific probes are selected as a probe set for identification of a target sequence.

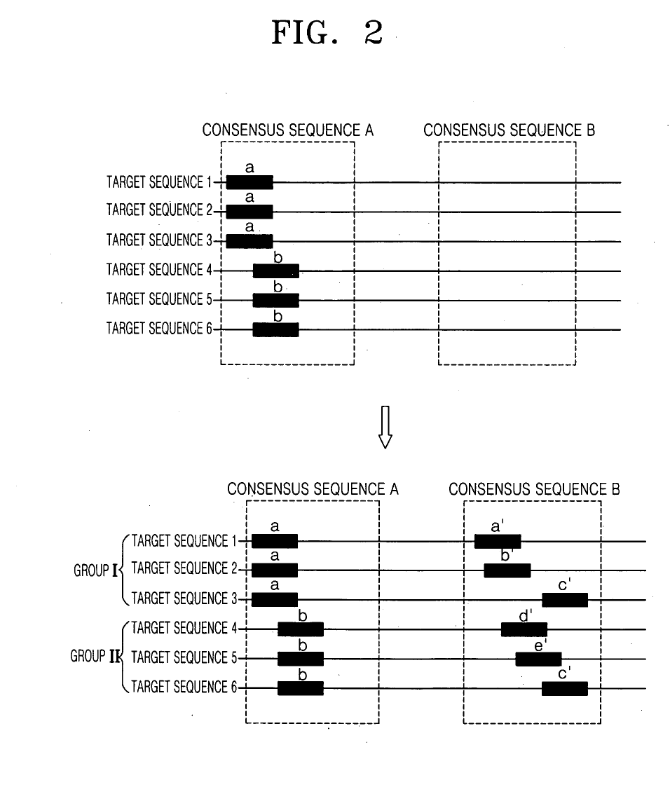

FIG. 2 is a schematic diagram of a method of designing a probe set using two consensus sequences according to an embodiment of the present invention.

Although 6 target sequences are used in this case, the number of target sequences is not restricted thereto. It will be understood by those skilled in the art that as the number of target sequences increases, the present invention is more potent.

Referring to FIG. 2, a consensus sequence A of 6 target sequences is compared to form a group I consisting of target sequences 1, 2, and 3, which contain a polynucleotide a, and a group II consisting of target sequences 4, 5, and 6, which contain a polynucleotide b. Since both the group I and the group II consist of two or more target sequences, the polynucleotides a and b are respectively selected as group probes of the groups I and II.

Then, another consensus sequence of target sequences of each of the groups I and II is compared. Since this operation is individually performed on each group, the consensus sequence used in the group I can be different from the consensus sequence in the group II. For example, a consensus sequence B can be compared in the group I and a consensus sequence C can be compared in the group II. Referring to FIG. 2 again, the consensus sequence B of the target sequences 1, 2, and 3 of the group I is compared to form a group I-1 consisting of the target sequence 1, which contains a polynucleotide a′, a group I-2 consisting of the target sequence 2, which contains a polynucleotide b′, and a group I-3 consisting of the target sequence 3, which contains a polynucleotide c′. Also, the consensus sequence B of the target sequences 4, 5, and 6 of the group II is compared to form a group II-1 consisting of the target sequence 4, which contains a polynucleotide d′, a group II-2 consisting of the target sequence 5, which contains a polynucleotide e′, and a group II-3 consisting of the target sequence 6, which contains a polynucleotide c′. Since all groups consist of one target sequence, the polynucleotides a′, b′, c′, d′, and e′ are selected as target sequence specific probes.

Although group probes of two target sequences are different from each other as in the case of the target sequences 3 and 6, target sequence specific probes thereof can be identical to each other.

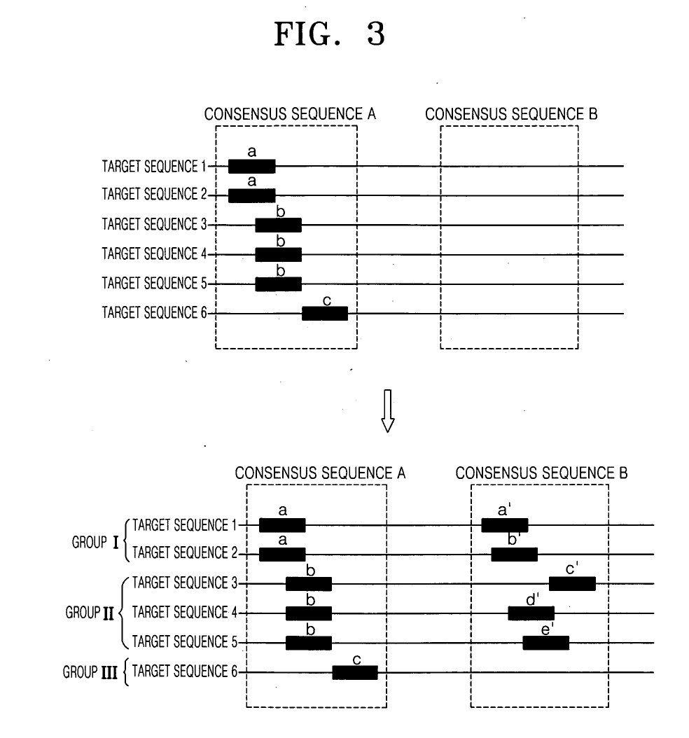

FIG. 3 is a schematic diagram of a method of designing a probe set using two consensus sequences according to another embodiment of the present invention.

Referring to FIG. 3, a consensus sequence A of 6 target sequences is compared to form a group I consisting of target sequences 1 and 2, which contain a polynucleotide a, a group II consisting of target sequences 3, 4, and 5, which contain a polynucleotide b, and a group III consisting of the target sequence 6, which contains a polynucleotide c. Since the group III consists of one target sequence, the polynucleotide c is selected as a target sequence specific probe of the target sequence 6. Meanwhile, since the groups I and II consist of two or more target sequences, the polynucleotides a and b are respectively selected as group probes of the groups I and II. Then, a consensus sequence B of the target sequences 1 and 2 of the group I is compared to form a group I-1 consisting of the target sequence 1, which contains a polynucleotide a′ and a group I-2 consisting of the target sequence 2, which contains a polynucleotide b′. Since the groups I-1 and I-2 consist of one target sequence, the polynucleotides a′ and b′ are respectively selected as target sequence specific probes of the target sequences 1 and 2. Similarly, the consensus sequence B of the target sequences 3, 4, and 5 of the group II is compared to form a group II-1 consisting of the target sequence 3, which contains a polynucleotide c′, a group II-2 consisting of the target sequence 4, which contains a polynucleotide d′, and a group II-3 consisting of the target sequence 5, which contains a polynucleotide e′. Since all the groups II-1, II-2, and II-3 consist of one target sequence, the polynucleotides c′, d′, and e′ are respectively selected as target sequence specific probes of the target sequences 3, 4, and 5.

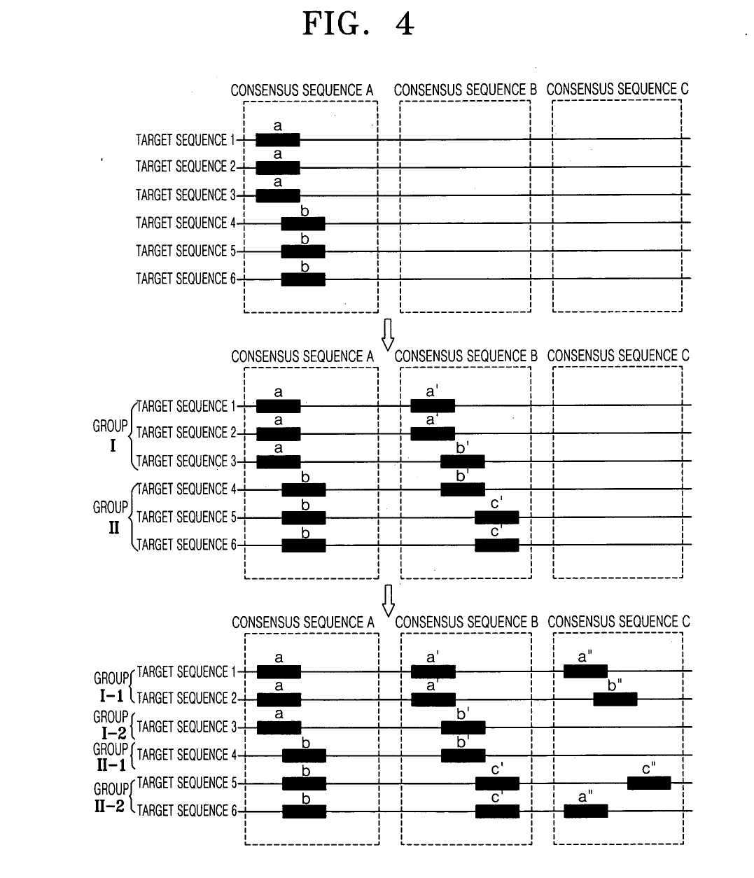

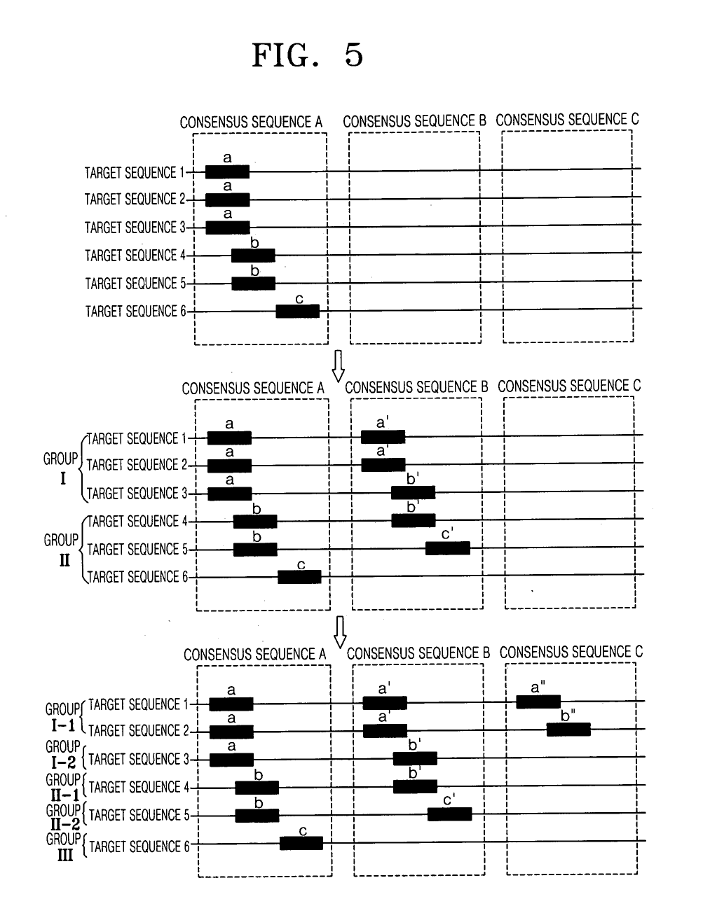

FIG. 4 is a schematic diagram of a method of designing a probe set using three consensus sequences according to an embodiment of the present invention and FIG. 5 is a schematic diagram of a method of designing a probe set using three consensus sequences according to another embodiment of the present invention.

Referring to FIGS. 4 and 5, when at least one group consists of two or more target sequences even after comparing two consensus sequences, a third consensus sequence of the group consisting of two or more target sequences can be compared. The comparison method is as described above. Although three consensus sequences are used in FIGS. 4 and 5, four or more consensus sequences can be used when the number of target sequences to be identified is very large.

According to another embodiment of the present invention, there is provided a probe set designed using the method described above.

According to another embodiment of the present invention, there is provided a microarray having a substrate on which the probe set is immobilized The microarray may be manufactured using the probe set according to a typical method known to those skilled in the art.

That is, the substrate may be coated with an active group selected from the group consisting of amino-silane, poly-L-lysine, and aldehyde. The substrate may be a silicon wafer, glass, quartz, metal, or plastic. The probe set may be immobilized on the substrate using a piezoelectric micropipetting method, a pin-shaped spotter, etc.

According to another embodiment of the present invention, there is provided a method of identifying target sequences using the probe set. The method of identifying target sequences may be performed using the microarray. The method of identifying target sequences may include: applying a sample including target sequences on the microarray; hybridizing the target sequences with the probe set; washing the microarray to remove a non-specific reaction; and detecting a fluorescent signal due to hybrid formation.

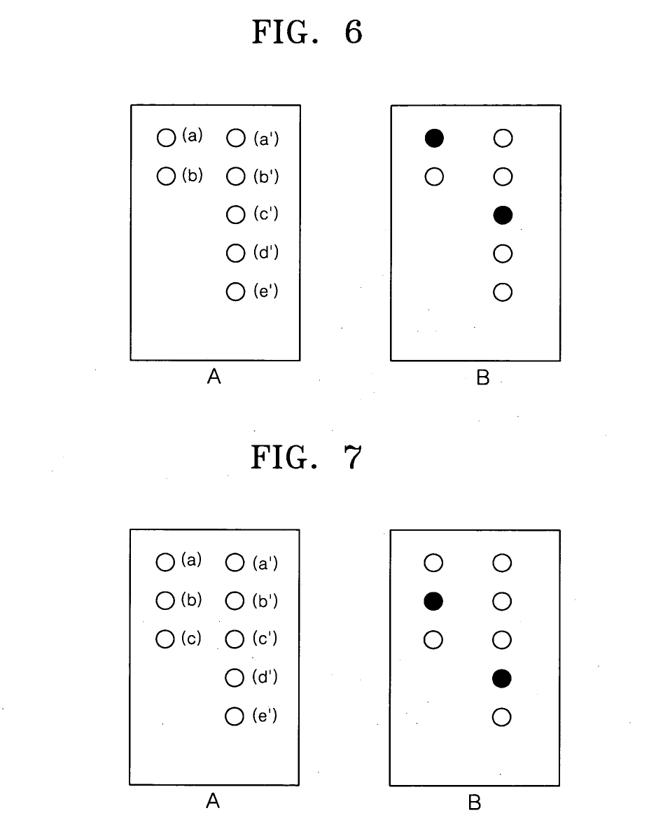

FIGS. 6 through 9 illustrate spotting arrangements of microarrays including probe sets designed according to methods illustrated in FIGS. 2 through 5 and methods of identifying a target sequence using the microarrays.

Although a probe set can be spotted on the microarray such that probes are separately arranged on the basis of a consensus sequence from which they are derived, as illustrated in FIGS. 6 through 9, the spotting arrangement is not particularly restricted.

Referring to FIG. 6, a microarray is manufactured by arranging the polynucleotides a and b, which are group probes derived from the consensus sequence A, in a column and arranging the polynucleotides a′, b′, c′, d′, and e′, which are target sequence specific probes derived from the consensus sequence B, in the other column (A). As a result of performing the method of identifying a target sequence of the present invention using the microarray manufactured above, hybridization is observed in the probes a and c′ (B). Referring to FIG. 2 again, the probe a indicates the group I and the probe c′ indicates the target sequence 3 of the group 1. Thus, it can be identified that the target sequence 3 is contained in the sample.

Similarly, referring to FIG. 7, a microarray is manufactured by arranging the polynucleotides a and b, which are group probes derived from the consensus sequence A, and the polynucleotide c, which is a target sequence specific probe, in a column and arranging the polynucleotides a′, b′, c′, d′, and e′, which are target sequence specific probes derived from the consensus sequence B, in the other column (A). As a result of performing the method of identifying target sequences of the present invention using the microarray manufactured above, hybridization is observed in the probes b and d′ (B). Referring to FIG. 3 again, it can be identified that the target sequence 4 is contained in the sample.

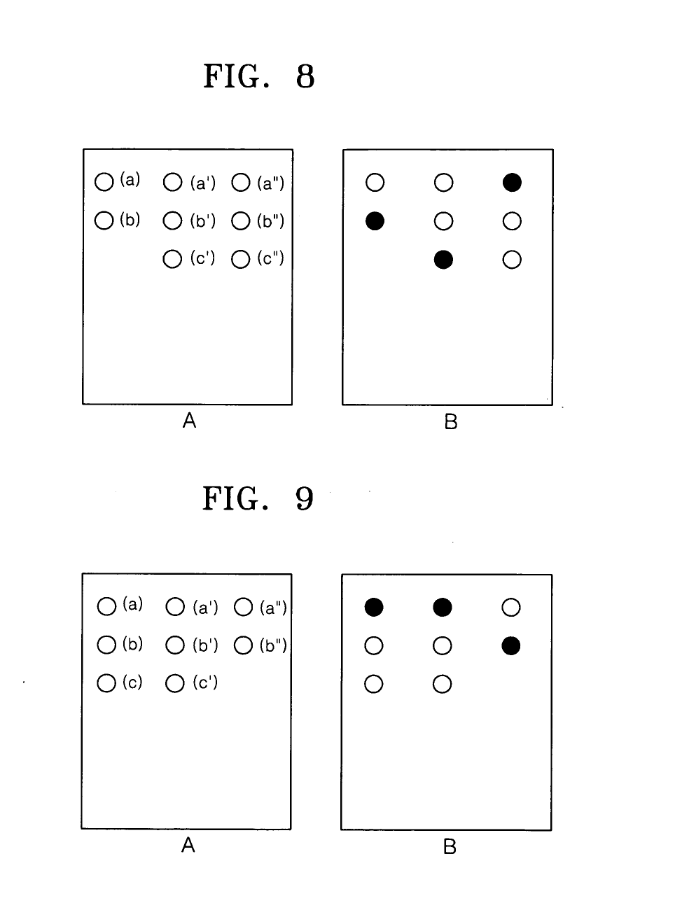

Referring to FIG. 8, a microarray is manufactured by arranging the polynucleotides a and b, which are group probes derived from the consensus sequence A, in a first column, the polynucleotides a′, b′, and c′, which are target sequence specific probes derived from the consensus sequence B, in a second column, and the polynucleotides a″, b″, and c″, which are target sequence specific probes derived from the consensus sequence C, in a third column (A). As a result of performing the method of identifying target sequences of the present invention using the microarray manufactured above, hybridization is observed in the probes b, c′, and a″ (B). Referring to FIG. 4 again, it can be identified that the target sequence 6 is contained in the sample.

Referring to FIG. 9, a microarray is manufactured by arranging the polynucleotides a, b, and c, which are group probes or target sequence specific probes derived from the consensus sequence A, in a first column, the polynucleotides a′, b′, and c′, which are group probes or target sequence specific probes derived from the consensus sequence B, in a second column, and the polynucleotides a″ and b″, which are target sequence specific probes derived from the consensus sequence C, in a third column (A). As a result of performing the method of identifying target sequences of the present invention using the microarray manufactured above, hybridization is observed in the probes a, a′, and b″ (B). Referring to FIG. 5 again, it can be identified that the target sequence 2 is contained in the sample.

According to another embodiment of the present invention, there is provided a computer readable medium recorded thereon a program to execute the method of designing a probe set.

The invention can also be embodied as computer (all devices with a data processing capability) readable codes on a computer readable recording medium. The computer readable recording medium is any data storage device that can store data which can be thereafter read by a computer system. Examples of the computer readable recording medium include read-only memory (ROM), random-access memory (RAM), CD-ROMs, magnetic tapes, floppy disks, optical data storage devices

In examples of the present invention, total 71 species of bacteria including 37 species related to sepsis and 34 species related to contamination during culturing blood or bacteremia were used to design a probe set capable of identify these bacteria. As a result, a probe set including 24 group probes and 56 target sequence specific probes which could identify 64 species of bacteria was designed.

The conventional method of designing a probe set using only 16S rRNA can identify only 35 species among 71 species of bacteria when it is designed so as to have a homology between probes of 90% or less and identify 12 species when it is designed so as to have a homology between probes of 80% or less. However, the method of the present invention can identify 64 species even when it is designed so as to have a homology between probes of 80% or less.

The present invention will be described in greater detail with reference to the following example. The following example is for illustrative purposes only, and is not intended to limit the scope of the invention.

### EXAMPLE 1

Design of a Probe Set for Identification of 71 Species of Bacteria

In the present example, total 71 species of bacteria including 37 species related to sepsis and 34 species related to contamination during culturing blood or bacteremia were used to design a probe set capable of identify these bacteria.

First, 16S rRNA sequence which was a consensus sequence of the 71 target species was compared to form groups, each of which consists of target species including 18-25 bp of polynucleotide which has a homology of 100% in the same group and 80% or less with polynucleotide of different group. The respective polynucleotides are selected as group probes of the respective groups. 16S rRNA sequence can vary according to species of strain and is available from a known sequence database, for example, GenBank. Examples of sequences are set forth with GenBank Accession No. in Table 1.

Next, 23S rRNA, sodA, gyrA, groEL or rpoB sequence was compared in two or more species of each group to form groups, each of which consists of target species including 18-25 bp of polynucleotide which has a homology of 100% in the same group and 80% or less with polynucleotide of different group. The formation of groups were performed until all groups consisted of one species and the respective polynucleotides were selected as species specific probes of the respective species. The consensus sequence of each species can vary according to species of strain and is available from a known sequence database, for example, GenBank.

Some of the designed group probes and species specific probes are set forth in Table 2. By the present example, a probe set including 24 group probes and 56 target sequence specific probes were designed. When using the probe set, 64 species except for 2 species (Bacteroides fragilis, Proteus penneri) of a low incidence related to sepsis and 5 species (Enterococcus gallinarum, Lactobacillus fermentum, Propionibacterium acnes, Corynebacterium Jeikeium, Aeromonas hydrophila) could be identified.

TABLE 1

| | | GenBank Accession No. of |
|---|---|---|
| No. | Species | 16S rRNA sequence |
| 1 | Bacteroides fragilis | NC_006347 |
| 2 | Clostridium perfringens | AB075767 |
| 3 | Chlamydophila pneumoniae | NC_005043 |
| 4 | Enterobacter aerogenes | AY186054 |
| 5 | Enterococcus avium | AY442814 |
| 6 | Enterococcus casseliflavus | AJ420804 |
| 7 | Enterobacter cloacae | AY736548 |
| 8 | Escherichia coli | NC_004431 |
| 9 | Enterococcus durans | AY683836 |
| 10 | Enterococcus faecium | AY723748 |
| 11 | Enterococcus faecalis | NC_004668 |
| 12 | Enterococcus raffinosus | AJ301838 |
| 13 | Enterobacter sakazakii | AY702097 |
| 14 | Haemophilus influenzae | NC_000907 |
| 15 | Klebsiella oxytoca | AJ630270 |
| 16 | Klebsiella pneumoniae | AY736552 |
| 17 | Listeria monocytogenes | NC_002973 |
| 18 | Mycobacterium avium | X74495 |
| 19 | Mycobacterium tuberculosis | AJ536031 |
| 20 | Neisseria gonorrhoeae | AF398329 |
| 21 | Neisseria meningitides | AY573194 |
| 22 | Pseudomonas aeruginosa | AY631058 |
| 23 | Proteus mirabilis | AJ605736 |
| 24 | Proteus penneri | AJ634474 |
| 25 | Proteus vulgaris | AY186048 |
| 26 | Rickettsia rickettsii | AY573599 |
| 27 | Streptococcus agalactiae | NC_004116 |
| 28 | Staphylococcus aureus | NC_002952 |
| 29 | Streptococcus bovis | AY327523 |
| 30 | Salmonella enteritidis | AY186056 |
| 31 | Staphylococcus epidermidis | AY728198 |
| 32 | Serratia marcescens | AY730005 |
| 33 | Streptococcus mitis | AY005045 |
| 34 | Streptococcus pneumoniae | NC_003098 |
| 35 | Streptococcus pyogenes | NC_004070 |
| 36 | Salmonella typhi | Z47544 |
| 37 | Yersinia enterocolitica | AJ639645 |
| 38 | Acinetobacter baumannii | Z93435 |
| 39 | Acinetobacter calcoaceticus | AY800383 |
| 40 | Aeromonas hydrophila | AB182089 |
| 41 | Acinetobacter lwoffii | Z93441 |
| 42 | Corynebacterium diphtheriae | BX248357 |
| 43 | Citrobacter freundii | AB182200 |
| 44 | Cardiobacterium hominis | AY360343 |
| 45 | Corynebacterium jeikeium | X84250 |
| 46 | Campylobacter jejuni | AY830883 |
| 47 | Enterococcus gallinarum | AY346316 |
| 48 | Fusobacterium nucleatum | AJ810282 |
| 49 | Haemophilus aphrophilus | AY362906 |
| 50 | Haemophilus parainfluenzae | AY362908 |
| 51 | Lactobacillus fermentum | AJ617543 |
| 52 | Micorococcus luteus | AB182215 |
| 53 | Morganella morganii | AB182240 |
| 54 | Propionibacterium acnes | AF076032 |
| 55 | Pseudomonas fluorescens | NC_005043 |
| 56 | Pseudomanas putida | AY789573 |
| 57 | Staphylococcus capitis | AY688039 |
| 58 | Staphylococcus cohnii | AJ717378 |
| 59 | Staphylococcus haemolyticus | AY688062 |
| 60 | Staphylococcus hominis | AJ717375 |
| 61 | Streptococcus intermedius | Z69040 |
| 62 | Stenotrophomonas maltophilia | AY826621 |
| 63 | Streptococcus oralis | AY281080 |
| 64 | Streptococcus salivarius | AY669233 |
| 65 | Streptococcus sanguinis | AY691542 |
| 66 | Staphylococcus saprophyticus | AY688090 |
| 67 | Staphylococcus simulans | AY688101 |
| 68 | Salmonella typhimurium | NC_003197 |
| 69 | Streptococcus vestibularis | AY581143 |
| 70 | Staphylococcus warneri | AY688106 |
| 71 | Staphylococcus xylosus | AY688109 |

TABLE 2

| | Group probe | | Species specific |
|---|---|---|---|
| Group | sequence | Species | probe sequence |
| I | SEQ ID NO: | Cardiobacterium hominis | SEQ ID NO: 14 |
| | 1 | | |
| II | SEQ ID NO: | Enterobacter aerogenes | SEQ ID NO: 15 |
| | 2 | Escherichia coli | SEQ ID NO: 16 |
| | | Enterobacter sakazakii | SEQ ID NO: 17 |
| | | Salmonella typhimurium | SEQ ID NO: 18 |
| | | Salmonella typhi | SEQ ID NO: 19 |
| | | Morganella morganii | SEQ ID NO: 20 |
| | | Pseudomonas aeruginosa | SEQ ID NO: 21 |
| | | Proteus mirabilis | SEQ ID NO: 22 |
| | | Proteus vulgaris | SEQ ID NO: 23 |
| | | Streptococcus | SEQ ID NO: 24 |
| | | intermedius | |
| | | Salmonella enteritidis | SEQ ID NO: 25 |
| | | Yersinia enterocolitica | SEQ ID NO: 26 |
| III | SEQ ID NO: | Pseudomonas fluorescens | SEQ ID NO: 27 |
| | 3 | Pseudomonas putida | SEQ ID NO: 28 |
| IV | SEQ ID NO: | Acinetobacter baumannii | SEQ ID NO: 29 |
| | 4 | cinetobacter | SEQ ID NO: 30 |
| | | calcoaceticus | |
| | | Acinetobacter lwoffii | SEQ ID NO: 31 |
| V | SEQ ID NO: | Haemophilus aphrophilus | SEQ ID NO: 32 |
| | 5 | Haemophilus influenzae | SEQ ID NO: 33 |
| VI | SEQ ID NO: | Enterobacter cloacae | SEQ ID NO: 34 |
| | 6 | Klebsiella oxytoca | SEQ ID NO: 35 |
| VII | SEQ ID NO: | Aeromonas hydrophila | SEQ ID NO: 36 |
| | 7 | | |
| VIII | SEQ ID NO: | Mycobacterium avium | SEQ ID NO: 37 |
| | 8 | ycobacterium | SEQ ID NO: 38 |
| | | tuberculosis | |
| IX | SEQ ID NO: | Neisseria gonorrhoeae | SEQ ID NO: 39 |
| | 9 | Neisseria meningitides | SEQ ID NO: 40 |
| | | Stenotrophomonas | SEQ ID NO: 41 |
| | | maltophilia | |
| X | SEQ ID NO: | Streptococcus bovis | SEQ ID NO: 42 |
| | 10 | Streptococcus mitis | SEQ ID NO: 43 |
| | | Streptococcus pyogenes | SEQ ID NO: 44 |
| XI | SEQ ID NO: | Staphylococcus aureus | SEQ ID NO: 45 |
| | 11 | Staphylococcus capitis | SEQ ID NO: 46 |
| | | Staphylococcus cohnii | SEQ ID NO: 47 |
| | | Staphylococcus | SEQ ID NO: 48 |
| | | epidermidis | |
| | | Staphylococcus | SEQ ID NO: 49 |
| | | saprophyticus | |
| | | Staphylococcus | SEQ ID NO: 50 |
| | | haemolyticus | |
| | | Staphylococcus hominis | SEQ ID NO: 51 |
| | | Staphylococcus | SEQ ID NO: 52 |
| | | simulans | |
| | | Staphylococcus warneri | SEQ ID NO: 53 |
| | | Staphylococcus xylosus | SEQ ID NO: 54 |
| XII | SEQ ID NO: | Enterococcus avium | SEQ ID NO: 55 |
| | 12 | Enterococcus durans | SEQ ID NO: 56 |
| | | Enterococcus faecalis | SEQ ID NO: 57 |
| | | Enterococcus | SEQ ID NO: 58 |
| | | raffinosus | |
| XIII | SEQ ID NO: | Streptococcus mitis | SEQ ID NO: 59 |
| | 13 | Streptococcus pyogenes | SEQ ID NO: 60 |
| | | Streptococcus | SEQ ID NO: 61 |
| | | agalactiae | |
| | | Streptococcus oralis | SEQ ID NO: 62 |
| | | Streptococcus | SEQ ID NO: 63 |
| | | pneumoniae | |
| | | Streptococcus | SEQ ID NO: 64 |
| | | salivarius | |
| | | Streptococcus | SEQ ID NO: 65 |
| | | sanguinis | |
| | | Septococcus | SEQ ID NO: 66 |
| | | vestibularis | |

According to the present invention, a probe set which can rapidly identify a number of target sequences and accurately identify target sequences even when two or more target sequences coexist in a sample can be readily designed.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method of designing a probe set for identification of a target sequence, the method comprising:
(a) comparing a consensus sequence of target sequences to form groups, each of which consists of target sequences which include a polynucleotide contained in the consensus sequence and meeting a predetermined criterion;
(b) selecting an oligonucleotide specifically binding to the polynucleotide meeting the predetermined criterion as a target sequence specific probe when one of the groups formed in the operation (a) consists of one target sequence;
(c) selecting an oligonucleotide specifically binding to the polynucleotide meeting the predetermined criterion as a group probe when one of the groups formed in the operation (a) consists of two or more target sequences; and
(d) performing operations (a) to (c) on the groups formed in the operation (a) consisting of two or more target sequences using a consensus sequence other than the consensus sequence used in the operation (a) until there are no groups consisting of two or more target sequences.

2. The method of claim 1, wherein the predetermined criterion is at least one selected from the group consisting of a sequence homology, a base length, a hybridization melting point (Tm), a difference between hybridization melting points (ΔTm), a GC content, self-alignment, a mutation position, a repeating sequence level, and a base composition at the 3′ end.

3. The method of claim 1, wherein the predetermined criterion is a homology of 100% for polynucleotides of the same group and 90% or less for polynucleotides of different groups.

4. The method of claim 1, wherein the consensus sequence is 16S rRNA, 23S rRNA, sodA, gyrA, groEL, or rpoB.

5. A probe set designed using the method of claim 1.

6. A microarray for identification of target sequences, in which the probe set of claim 5 is immobilized on a substrate.

7. The microarray of claim 6, wherein the substrate is coated with an active group selected from the group consisting of amino-silane, poly-L-lysine, and aldehyde.

8. The microarray of claim 6, wherein the substrate is a silicon wafer, glass, quartz, metal, or plastic.

9. A computer readable medium recorded thereon a program to execute the method of claim 1.

10. A method of identifying target sequences using the probe set of claim 5.

11. A method of identifying target sequences using the probe set of claim 5, which comprises:
applying a sample including target sequences on the microarray for identification of target sequences, in which the probe set of claim 5 is immobilized on a substrate;
hybridizing the target sequences with the probe set;
washing the microarray to remove a non-specific reaction; and
detecting a fluorescent signal due to hybrid formation.
